(19) United States
(12) Patent Application Publication
(10) Pub. No.: US 2023/0181349 A1
(43) Pub. Date: Jun. 15, 2023

(54) DENTAL APPARATUS FOR HOLDING THE TONGUE IN POSITION BETWEEN THE TEETH AND METHOD FOR USING SAME

(71) Applicant: Guillermo Reyes, SAN ANTONIO, TX (US)

(72) Inventor: Guillermo Reyes, SAN ANTONIO, TX (US)

(21) Appl. No.: 17/550,532

(22) Filed: Dec. 14, 2021

(51) Int. Cl. A61F 5/56

(52) U.S. Cl. CPC A61F 5/566

(57) ABSTRACT

A dental appliance is designed to hold the tongue in position between the teeth, and can hold the jaw jutted forward as well, during sleep in order to lessen the occurrence of snoring. The method of the present invention involves placing the dental appliance over the teeth in the mouth, gripping the tongue between the teeth with the dental appliance, and going to sleep. The dental appliance is intended to reduce snoring and can be used to treat obstructive sleep apnea.

### BACKGROUND OF THE INVENTION

### Field of the Invention

Applicant’s invention relates to a device for holding the tongue in position between the teeth and method for using same. More particularly, it relates to a mouthpiece that can be used to hold the tongue forward from the throat in order to reduce snoring.

### Background Information

Snoring is a rattling noise made during sleep by vibration of the soft palate (the back of the roof of the mouth) and the uvula (the prominent structure dangling down at the back of the mouth). When a person breathes, air is pulled into the lungs and then pushed back out. To get into the lungs, the inspired air moves into the body via the nose or mouth and then past the tongue, the soft palate, the uvula, and the tonsils. When awake, people do not snore because the muscles in the back of the throat tighten to hold these structures in place and prevent them from collapsing and vibrating in the airway.

During sleep however, muscles in the body, including those in the back of the throat, tend to loosen. Thus, one mechanism that causes snoring happens when a patient dozes off and begins to progress from a light sleep to a deep sleep, the muscles in the roof of the mouth (soft palate), tongue and throat relax allowing the base of the tongue and soft palate collapse to the back wall of the throat. A patient lying on their back makes this more likely. Air passing by due to breathing, combined with the relaxed and collapsed the soft palate and uvula, causes them to vibrate resulting in snoring.

Less specifically, snoring happens when a person can’t move air freely through their nose and throat during sleep. People who snore often have too much throat and nasal tissue or “floppy” tissue that is more prone to vibrate. For various reasons, the tissue moves into the airway, causing the airway to be partially blocked while asleep. The sound is caused by tissues at the top of the airway striking each other. Thus, the surrounding tissues vibrate, which produces the familiar snoring sound. When severe, snoring can cause disruptions in sleep for both the snorer and others that may be nearby. Snoring is common, especially among older people and people who are overweight.

Snoring can be caused by a number of factors, such as the mouth and sinuses’ anatomy, alcohol consumption, allergies, illnesses such as a cold, and the patient’s weight - all of which can cause or contribute to airway blockage. Snoring can also be a sign of a serious sleep disorder called sleep apnea.

It is estimated that approximately 45% of adults snore at least occasionally. For a few of those snoring is only a nuisance, but 75% of people who snore have obstructive sleep apnea (when breathing is disrupted during sleep for short periods), which increases the risk of developing heart disease.

There are many techniques for self-treating snoring, such as changing sleep position from the back to the side, losing weight (although thin people snore, too), avoiding alcohol and sedatives which can reduce the resting tone of the muscles in the back of the throat, keeping nasal passages open, reduce allergens, staying well hydrated which can help keep secretions in the nose and soft palate less sticky. There are also many devices designed to help reduce snoring including mouthpieces. Conventional anti-snoring mouthpieces combat snoring by fitting snugly around the teeth with the lower portion moving the jaw forward relative to its normal position and preventing the jaw from falling back while asleep. This helps open the airway in the back of the throat so air can pass more easily without causing snoring.

### SUMMARY OF THE INVENTION

The present invention is a dental appliance that is designed to hold the tongue in position between the teeth during sleep in order to lessen the occurrence of snoring. The method of the present invention involves placing the dental appliance over the teeth in the mouth, gripping the tongue between the teeth with the dental appliance, and going to sleep. The dental appliance is intended to reduce snoring and can be used to treat obstructive sleep apnea.

The dental appliance, or mouthpiece, fits onto the teeth. It has an upper trough that fits over the upper teeth, and a lower ridge that fits over the lower teeth. Fitting in the back of the mouthpiece is a pivot that connects the back portions of the trough and ridge together. There is an aperture between the trough and ridge that is sized to accept the tongue of the wearer. The wearer urges the mouthpiece trough onto the wearer’s top teeth and urges the ridge onto the wearer’s bottom teeth. The wearer then inserts the wearer’s tongue through an aperture in the mouthpiece between the trough and the ridge, and bites down on the tongue. The dental appliance helps hold the tongue. The tongue is held in an extended position by the pressure with the teeth and mouthpiece. In order to not damage the tongue, the mouthpiece is made of a soft plastic.

“Soft,” as used herein, describes a measure of the resistance to localized plastic deformation induced by either mechanical indentation. Softness is dependent on ductility, elastic stiffness, plasticity, strain, strength, toughness, viscoelasticity, and viscosity. Indentation hardness measures the resistance of a sample to material deformation due to a constant compression load from a sharp object. The Durometer is the name of the scale that is used to measure the level of hardness and durability of plastics, silicon, rubber, and like materials. A common scale of measure for the durometer is the “Shore” system which assigns a letter “A” for softer materials, and a letter “D” for harder materials. The scales follow the American Society for Testing and Materials (ASTM), in the use of a 0-100 range to define the softness or hardness of a given material. It is anticipated that the mouthpiece would be Shore A 50, or softer.

In one embodiment, it is anticipated that the mouthpiece would be in the range of Shore A 15 to Shore A 25. The mouthpiece is made from softer material in order to avoid the teeth damaging the tongue when pressure is applied.

It is anticipated that in another embodiment, the mouthpiece would be made from materials from 2 or more durometer ratings of softness. In this embodiment, at least the portion of the mouthpiece in contact with the tongue (the upper and lower grip surfaces) would be Shore A 50, or softer, and could be in the range of Shore A 15 to Shore A 25. It is anticipated that the remainder of the mouthpiece could be made from a harder materials in order to provide improved structural integrity and/or to extend the useful life of the mouthpiece.

The mouthpiece holds the tongue forward which counter-acts having the tongue slouch backward into the mouth and obstructing the airway. Thus, the mouthpiece increases the size of the airway (or keeps it from being blocked) in order to reduce snoring.

It is easy to release the grip on the tongue so that the tongue is free to be removed from the aperture in the mouthpiece and from between the front teeth. Once released, the mouthpiece is removable from the teeth and mouth and can be cleaned outside the mouth with soap and water, or other cleansing materials. Additionally, when a mouthpiece becomes too damaged due to use, it can simply be disposed of normally.

In addition to holding the tongue extended, the mouthpiece can provide a combination of holding the tongue and moving the jaw forward so that the airway is maximized. The mouthpiece can help hold the jaw because the trough and ridge fit securely around the teeth and the pivot can be made or adjusted such that the jaw extends. It is anticipated that the ridge will be offset forward of the trough in the range of 0.25 cm - 1.0 cm. For example, an embodiment of the dental appliance may have the ridge offset 0.5 cm forward from the trough, or the device sets the bottom jaw 0.5 cm forward from the front teeth.

The pivot portion at the back end of the mouthpiece can help reduce the person grinding their teeth while sleeping.

The dental appliance can be made in different sizes to help accommodate the different sizes of mouths and teeth.

Thus, the dental appliance is designed to increase the airway size by both moving the tongue forward and moving the jaw forward so that the airway is maximized.

### BRIEF DESCRIPTION OF THE DRAWINGS

FIG. 1 is a perspective view of the dental appliance.

FIG. 2 is a side view of the dental appliance as placed in the mouth when in use.

FIG. 3 is a front view of the dental appliance as placed in the mouth when in use.

### DETAILED DESCRIPTION OF THE PREFERRED EMBODIMENT

| Ref. | Element | Ref. | Element |
|---|---|---|---|
| 10 | Mouthpiece | 34 | Upper Grip Surface |
| 12 | Trough | 36 | Pivot |
| 14 | Ridge | 38 | Vertices Line |
| 16 | Outer Side | 40 | Back Edge |
| 18 | Outer Side Rim | 42 | Contour |
| 20 | Floor | 100 | Tongue |
| 22 | Inner Side | 102 | Upper Teeth |
| 24 | Inner Side Rim | 104 | Lower Teeth |
| 26 | Outer Wall | 106 | Mouth |
| 28 | Lower Grip Surface | 108 | Jaw |
| 30 | Inner Wall | 110 | Face |
| 32 | Aperture | 112 | Skull |

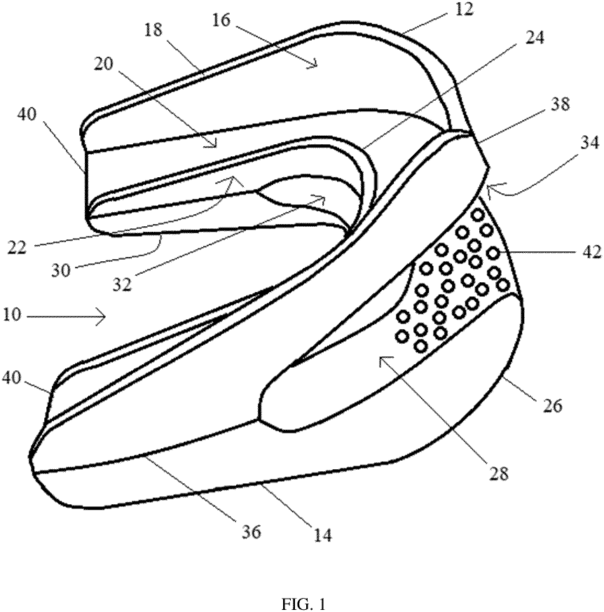

Referring to the figures, FIG. 1 illustrates of the dental appliance. For purposes of clarity, when directions are referred to herein such as up and down, upper and lower, inside and outside, and the like, they are referring to those positions when the mouthpiece 10 is in place on a person’s teeth (102 & 104) and the person is standing where “up” is up from the ground, “back” is back toward the person’s throat, “outside” is on the outside of the teeth (102 & 104), and other directions are based on these standards.

The dental appliance, or mouthpiece, 10 is comprised of two main portions, the upper trough 12 and the lower ridge 14. The trough 12 is comprised of an outer side 16 that extends from an outer side rim 18 down to where it connects to the outside edge of the floor 20. And inner side 22 extends downwardly from an inner side rim 24 down to where the inner side 22 connects to the inside edge of the floor 20. Thus, the trough 12 has a half-pipe shape from outer side 16 to floor 20 to inner side 22. The trough 12 is a generally U-shaped from the front vertices line 38 from where it bends backward on each side to a back edge 40. The trough 12 is shaped and sized such that it can be urged over the top teeth 102 and fits snugly.

The lower ridge 14 is comprised of an outer wall 26 that extends downwardly from where it connects to the outer edge of the lower grip surface 28. And inner wall 30 extends downwardly from where it connects to the inner edge of the lower grip surface 28. Thus, the ridge 14 has an inverted half-pipe shape from outer wall 26 to lower grip surface 28 to inner wall 30. The ridge 14 is generally U-shaped from the front vertices line 38 from where it bends backward on each side to a back edge 40, thus basically mirroring the upper U-shape. The ridge 14 is shaped and sized such that it can be urged over the lower teeth 104 and fits snugly.

Regarding the trough 12, outer side 16 is designed to fit on the outside of the upper teeth 102. The outer side 16 extends generally upwardly when the mouthpiece 10 is positioned in the mouth 106. The floor 20 fits against the bottom of the upper teeth 102. The inner side 22 is designed to fit on the inside of the upper teeth 102, and extending upwardly.

Conversely, regarding the ridge 14, outer wall 26 is designed to fit on the outside of the lower teeth 104. The outer wall 26 extends generally downwardly when the mouthpiece 10 is positioned in the mouth 106. The lower grip surface 28 rests on the top of the lower teeth 104.

The trough 12 is connected to the ridge 14 at or near the back edges 40. The upper grip surface 34 (which is the underside of the floor 20) is connected to the upper surface (opposite the surface that contacts the lower teeth 104) of the lower grip surface 28. The connection of trough 12 to ridge 14 extends along pivot 36. There are many embodiments that pivot 36 may take, such as molded together, glued together, a hinge, or other connector.

At the front of the pivot 36 the trough 12 and ridge 14 separate to create an aperture 32. The aperture 32 is designed, sized and received, in order to accept the wearer’s tongue 100 there through. When the mouthpiece 10 is positioned on the upper teeth 102 and lower teeth 104, the person may extend their tongue through the aperture 32 and then bite down with enough force such that the tongue 100 is held in place. Along with the pressure, the softness of the lower grip surface 28 and upper grip surface 34 helps hold the tongue 100. Additionally, the lower grip surface 28 and/or the upper grip surface 34 may have contours 42 in order to help hold the tongue 100. The contours 42 may take the form of bumps, grooves, ridges, or other structures that can increase the coefficient of friction of the lower grip surface 28 and upper grip surface 34.

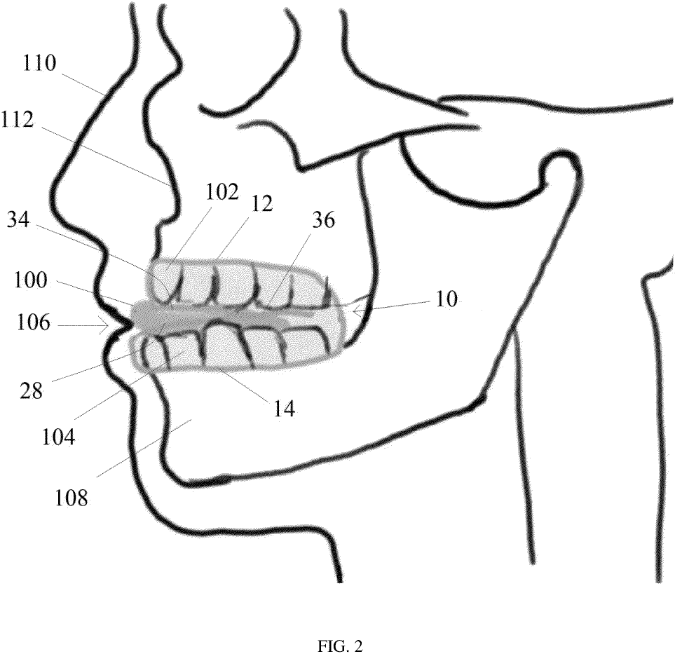

FIG. 2 shows a side view of the dental appliance 10 as placed in the mouth 106 when in use. The face 110 is cutaway in order to better show the positioning of the mouthpiece 10. The mouthpiece 10 is shown in position, the trough 12 urged snugly over the upper teeth 102, and the ridge 14 urged snugly over the lower teeth 104. The tongue 100 is inserted through the aperture 32 and held between the lower grip surface 28 and the upper grip surface 34 due to pressure exerted by the jaw 108 against the skull 112. The ability of the mouthpiece 10 to hold the tongue 100 in place is improved due to the softness of the lower grip surface 28 and upper grip surface 34, and may be further improved with the addition of contours 42 on the lower grip surface 28 and upper grip surface 34.

As is clarified here, the trough 12 and ridge 14 are connected along the pivot 36. As described above, the vertices line 38 of ridge 14 may be offset slightly forward of the diversity line 38 of trough 12. Because the mouthpiece 10 is snugly fit to the upper teeth 102 and lower teeth 104, the offset of the ridge 14 tends to pull the jaw 108 forward as compared to its normal position in relation to the skull 112. In effect, this creates a bit of a under bite and helps keep the person’s airway open.

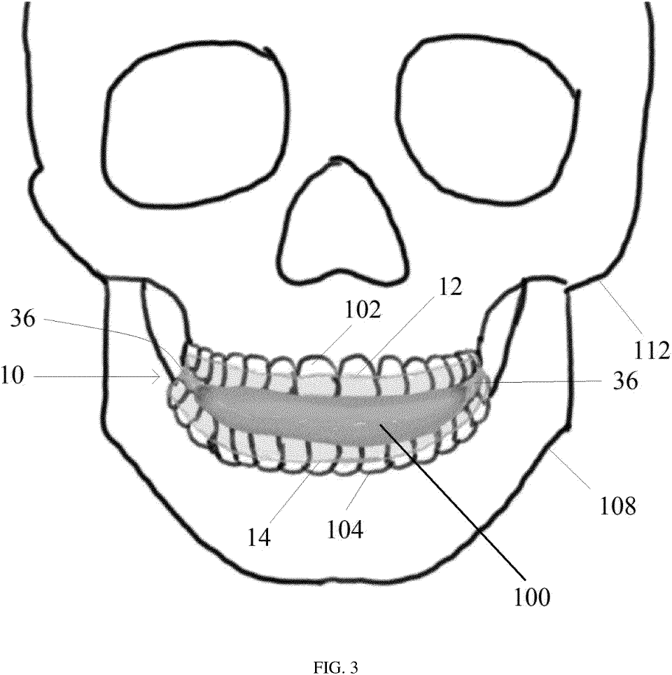

FIG. 3 shows a front view of the dental appliance 10 as placed in the mouth 100 when in use. In this view, the tongue 100 is shown inserted through the aperture 32 and held extended outwardly from the skull 112 and jaw 108. The trough 12 is shown in place over the upper teeth 102 and likewise, the ridge 14 is shown in place over the lower teeth 104. The pivots 36 are shown toward the back of the teeth (102 & 104) and mouth 106. This figure illustrates how the connection of the trough 12 and ridge 14 at the first pivot 36 with the trough 12 and ridge 14 separating and extending around until they again join at the second pivot 36, define the aperture 32 through which the tongue 100 is inserted and held.

Unless otherwise specifically noted, articles depicted in the drawings are not necessarily drawn to scale, however the drawings are illustrative and do indicate relative size and placement.

Throughout this disclosure, a hyphenated form of a reference numeral refers to a specific instance of an element and the un-hyphenated form of the reference numeral refers to the element generically or collectively. Thus for example, widget 12-1 would refer to a specific widget of a widget class 12, while the class of widgets may be referred to collectively as widgets 12 and any one of which may be referred to generically as a widget 12.

When the terms “substantially,” “approximately,” “about,” or “generally” are used herein to modify a numeric value, range of numeric values, or list numeric values, the term modifies each of the numerals. Unless otherwise indicated, all numbers expressing quantities, units, percentages, and the like used in the present specification and associated claims are to be understood as being modified in all instances by the terms “approximately,” “about,” and “generally.” As used herein, the term “approximately” encompasses +/-5 of each numerical value. For example, if the numerical value is “approximately 80,” then it can be 80 +/-5, equivalent to 75 to 85. As used herein, the term “about” encompasses +/-10 of each numerical value. For example, if the numerical value is “about 80,” then it can be 80 +/-10, equivalent to 70 to 90. As used herein, the term “generally” encompasses +/-15 of each numerical value. For example, if the numerical value is “about 80,” then it can be 80% +/-15, equivalent to 65 to 95. Accordingly, unless indicated to the contrary, the numerical parameters (regardless of the units) set forth in the following specification and attached claims are approximations that may vary depending upon the desired properties sought to be obtained by the exemplary embodiments described herein. In some ranges, it is possible that some of the lower limits (as modified) may be greater than some of the upper limits (as modified), but one skilled in the art will recognize that the selected subset will require the selection of an upper limit in excess of the selected lower limit.

At the very least, and not limiting the application of the doctrine of equivalents to the scope of the claim, each numerical parameter should at least be construed in light of the number of reported significant digits and by applying ordinary rounding techniques.

The terms “inhibiting” or “reducing” or any variation of these terms refer to any measurable decrease, or complete inhibition, of a desired result. The terms “promote” or “increase” or any variation of these terms includes any measurable increase, or completion, of a desired result.

The term “effective,” as that term is used in the specification and/or claims, means adequate to accomplish a desired, expected, or intended result.

The terms “a” or “an” when used in conjunction with the term “comprising” in the claims and/or the specification may mean “one,” but it is also consistent with the meaning of “one or more,” “at least one,” and “one or more than one.”

The term “each” refers to each member of a set, or each member of a subset of a set.

The term “removable” herein means that the removable part is designed and intended to be moved from its place or position in regard to another piece or pieces, and that unless otherwise noted, the removal of the part will not damages the piece or pieces. Further, it is presumed that, unless otherwise noted, if a part is removable it is also replaceable back into its original place or position in regard to the piece or pieces without damage to them.

The terms “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

In interpreting the claims appended hereto, it is not intended that any of the appended claims or claim elements invoke 35 U.S.C. 112(f) unless the words “means for” or “step for” are explicitly used in the particular claim.

It should be understood that, although exemplary embodiments are illustrated in the figures and description, the principles of the present disclosure may be implemented using any number of techniques, whether currently known or not. The present disclosure should in no way be limited to the exemplary implementations and techniques illustrated in the drawings and description herein. Thus, although the invention has been described with reference to specific embodiments, this description is not meant to be construed in a limited sense. Various embodiments may include some, none, or all of the enumerated advantages. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention. Modifications, additions, or omissions may be made to the systems, apparatuses, and methods described herein without departing from the scope of the disclosure. For example, the operations of the systems and apparatuses disclosed herein may be performed by more, fewer, or other components in the methods described may include more, fewer, or other steps. Additionally, steps may be performed in any suitable order.

I claim:

1. A mouthpiece to place on a person’s upper and lower teeth, the mouthpiece for holding a tongue comprising:
a trough, said trough further comprising an outer side, a floor, wherein an outer edge of said floor is connected to a lower edge of said outer side, and an inner side, wherein an inner edge of said floor is connected to a lower edge of said inner side such that a cross section of said trough is half-pipe shaped, and wherein said trough is U shaped and sized to fit snugly over said upper teeth;
a ridge, said ridge further comprising an outer wall, a lower grip surface, wherein an outer edge of said lower grip surface is connected to an upper edge of said outer wall, and an inner wall, wherein an inner edge of said lower grip surface is connected to an upper edge of said inner wall such that a cross section of said ridge is inverted half-pipe shaped, and wherein said ridge is U shaped and sized to fit snugly over said lower teeth;
wherein said trough is connected to said ridge by a pivot at or near each of two back edges;
wherein an aperture is defined between said trough and said ridge, and said two pivots, and wherein said aperture is sized to have said person’s tongue through said aperture; and
wherein said lower grip surface and said upper grip surface each have a softness in the range of Shore A 50 or less.

2. The mouthpiece of claim 1, wherein said lower grip surface and said upper grip surface each have a softness in the range of Shore A 15 to Shore A 25.

3. The mouthpiece of claim 1, wherein said ridge is offset forward of said trough in the range of 0.25 cm - 1.0 cm.

4. The mouthpiece of claim 3, wherein said lower grip surface and said upper grip surface each have a softness in the range of Shore A 15 to Shore A 25.

5. The mouthpiece of claim 1, wherein said ridge is offset forward of said trough by approximately 0.5 cm.

6. The mouthpiece of claim 5, wherein said lower grip surface and said upper grip surface each have a softness in the range of Shore A 15 to Shore A 25.

7. The mouthpiece of claims 1-6, further comprising a contour, wherein said contour is located on said lower grip surface or said upper grip surface.
